(19) United States
(12) Patent Application Publication
(10) Pub. No.: US 2016/0122315 A1
(43) Pub. Date: May 5, 2016

(54) CRYSTALLINE IMATINIB MESYLATE PROCESS

(71) Applicant: SHILPA MEDICARE LIMITED, Raichur (IN)

(72) Inventors: BHAGAT RAJ PIPAL, RAICHUR (IN); VEERESH APPA, RAICHUR (IN); MANISH SHARMA, RAICHUR (IN); AKSHAY KANT CHATURVEDI, RAICHUR (IN)

(73) Assignee: SHILPA MEDICARE LIMITED, RAICHUR, OT (IN)

(21) Appl. No.: 14/891,325

(22) PCT Filed: Apr. 4, 2014
(86) PCT No.: PCT/IB2014/060430

§ 371 Date: Nov. 14, 2015

(30) Foreign Application Priority Data
Jun. 12, 2013 (IN) 2548/CHE/2013

(51) Int. Cl. C07D 401/04; C07C 303/32

(52) U.S. Cl. CPC C07D 401/04; C07C 303/32; C07B 2200/13

(57) ABSTRACT

The present invention relates to a process for preparation of crystalline non-needle shaped Form-SA of Imatinib mesylate (I).

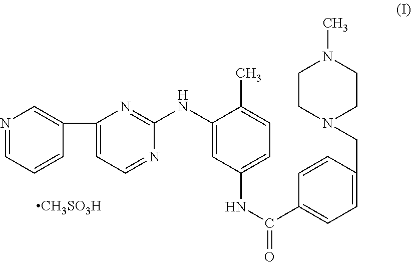

The invention also relates to crystalline Form-SA obtained by the process of the present invention, the said Form-SA being substantially pure, stable, non-needle shaped and characterized by X-ray powder diffraction pattern comprising of at least five 2θ° peaks selected from 10.67, 12.90, 15.34, 19.49, 19.80, 26.06, 26.32 and 28.89±0.05 2θ°.

The invention further relates to pharmaceutical compositions comprising non-needle shaped crystalline Form-SA of Imatinib mesylate, useful for the treatment of cancer.

### FIELD OF THE INVENTION

The present invention relates to a process for preparation of crystalline non-needle shaped Form-SA of Imatinib mesylate (I).

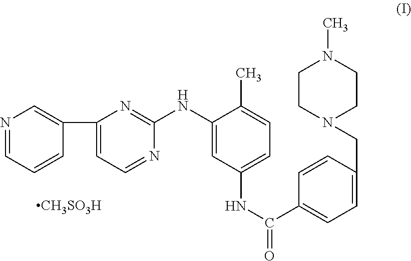

The invention also relates to crystalline Form-SA obtained by the process of the present invention, the said Form-SA being substantially pure, stable, non-needle shaped and characterized by X-ray powder diffraction pattern comprising of at least five 2θ° peaks selected from 10.67, 12.90, 15.34, 19.49, 19.80, 26.06, 26.32 and 28.89±0.05 2θ°.

The invention further relates to pharmaceutical compositions comprising non-needle shaped crystalline Form-SA of Imatinib mesylate, useful for the treatment of cancer.

### INTRODUCTION

Imatinib mesylate (I) is chemically known as 4-[(4-Methyl-1-piperazinyl)methyl]-N-[4-methyl-3-[[4-(3-pyridinyl)-2-pyrimidinyl]amino]-phenyl]benzamide methanesulfonate.

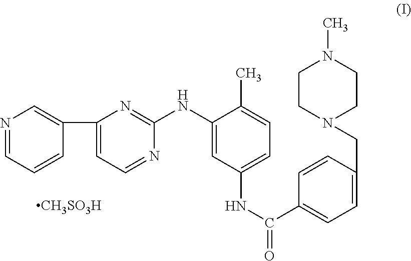

Imatinib mesylate is a tyrosine-kinase inhibitor used in the treatment of multiple cancers and is sold under the trade name GLEEVEC/GLIVEC®. Imatinib is used in chronic myelogenous leukemia (CML), gastrointestinal stromal tumors (GISTs) and a number of other malignancies. The U.S. Food and Drug Administration (FDA) has approved Imatinib as first-line treatment for Philadelphia chromosome (Ph)-positive Chronic myelogenous leukemia (CML), both in adults and children. The drug is approved in multiple Ph-positive cases CML, including after stem cell transplant, in blast crisis, and newly diagnosed. The FDA first granted approval for advanced GIST patients in 2002. Later in 2012, Imatinib was approved also for use after the surgical removal of KIT-positive tumors to help prevent recurrence. The drug is also approved in unresectable KIT-positive GISTs.

Further FDA has approved imatinib for use in adult patients with relapsed or refractory Ph-positive Acute lymphoblastic leukemia (ALL), myelodysplastic/myeloproliferative diseases associated with platelet-derived growth factor receptor gene re-arrangements, aggressive systemic mastocytosis (ASM) without or an unknown D816V c-KIT mutation, hypereosinophilic syndrome (HES) and/or chronic eosinophilic leukemia (CEL) who have the FIP1L1-PDGFRα fusion kinase (CHIC2 allele deletion) or FIP1L1-PDGFRα fusion kinase negative or unknown, unresectable, recurrent and/or metastatic dermatofibrosarcoma protuberans. Recently on 25 Jan. 2013, Gleevec has been approved for use in children with Ph+ALL.

Zimmermann et al in EP 564409 Al initially disclosed the preparation of Imatinib in free form (not as a salt). Further Zimmermann et al in U.S. Pat. No. 6,894,051 B1 described α and β crystal forms of Imatinib Mesylate.

Zimmermann et al in U.S.'051 disclosed that the a-crystal form of 4-(4-methylpiperazin-1 -ylmethyl)-N-[4-methyl-3-(4-pyridin-3-yl) pyrimidin-2 -ylamino) phenyl]benzamide methane sulfonate i.e. Imatinib mesylate is characterized by needle-shaped crystals and is hygroscopic. It mentioned that in this form, the crystals are not particularly well-suited to pharmaceutical formulation as solid dosage forms, because their physical properties, for example their flow characteristics, are unfavorable.

It was further emphasized that under certain conditions, however, it is possible to obtain 4-(4-methylpiperazin-1-ylmethyl)-N-[4-methyl3-(4-pyridin-3-yl) pyrimidin-2-yl amino) phenyl]benzamide methane sulfonate in a crystal form which is not needle-shaped, this crystal form being described as β-crystal form. The β-crystal form of Imatinib mesylate is detailed as having the advantage of its flow properties being substantially more favorable than those of the a-crystal form. This crystal form has the further advantage of being thermodynamically more stable at temperatures below 140° C. Also applicant of U.S.'051 describes that the β-crystal form is less hygroscopic than the α-crystal form and thus also stores better and is easier to process.

Besides α and β crystal forms various other polymorphic forms of Imatinib mesylate and the process for preparation thereof have been described in patent publications WO2004/106326, WO2005/095379, WO 2006/054314, WO2006/0223816, WO2006/048890, WO2007/023182, WO2007/059963 and WO2011/108953.

Imatinib mesylate being an important anticancer therapeutic agent, additional and improved ways of preparing Imatinib mesylate salt may be of immense value to pharmaceutical science and the healthcare of cancer patients. Further exploring new forms of pharmaceutically active / useful compounds such as Imatinib mesylate may provide an opportunity to improve the drug performance characteristics of such products. Hence, there exists a need for the further development of new stable crystalline form of Imatinib mesylate and economically viable processes for its preparation, which may be commercially up scalable, safer for handling, less time consuming and with better and consistent quality parameters.

The inventors of this application have developed a process which provides a stable polymorphic crystalline form of Imatinib mesylate, designated as Form-SA, which is non-hygroscopic, non-needle shaped and thus has easy handling properties. The process of this invention provides the crystalline Form-SA of Imatinib mesylate in a substantially pure form, which is without any detectable impurities/contamination of any other previously known crystalline forms of Imatinib mesylate.

### SUMMARY OF INVENTION

Particular aspects of the present invention relate to a process for preparation of crystalline non-needle shaped Form-SA of Imatinib mesylate (I). Crystalline Form-SA of Imatinib mesylate obtained by the process of the present invention is found to be substantially pure, stable and non-needle shaped.

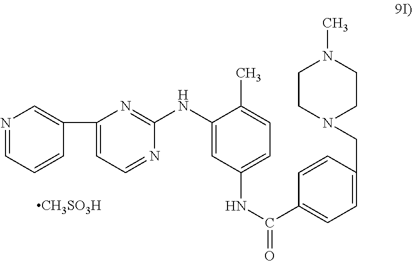

In one aspect according to the present invention, it provides process for the preparation of crystalline non-needle shaped Form-SA of Imatinib mesylate (I), comprising the steps of:
a) Providing a solution of Imatinib base in the mixture of isopropyl alcohol and a cyclic hydrocarbon or ether solvent;
b) Stirring the reaction mass at RPM of not less than 100 rotations per minute;
c) Adding solution of methane sulfonic acid and isopropyl alcohol to the reaction mass in time duration of not less than 30 mins;
d) Heating the reaction mass to a temperature ranging between 60-75° C.;
e) Cooling the reaction mass to ambient temperature of 25-30° C. in time duration of not less than 4 hours.
f) Recovering the non-needle shaped crystalline Form-SA.

In another aspect, the present invention provides crystalline non-needle shaped Form-SA of Imatinib mesylate, which is characterized by
i. X-ray powder diffraction pattern comprising of at least five 2θ° peaks selected from 10.67, 12.90, 15.34, 19.49, 19.80, 26.06, 26.32 and 28.89±0.05 2θ°;
ii. X-ray powder diffraction pattern with absence of 2θ° peak at about 25.08 2θ°;
iii. Non-needle shaped crystals;
iv. Particle size distribution of d₉₀ ranging from 90 to 130 μm.

In another aspect, crystalline non-needle shaped Form-SA of Imatinib mesylate obtained by the process of the present invention is having HPLC purity of at least 99.8% and moisture content of less than 0.5%. The crystalline Form-SA of Imatinib mesylate as obtained by the process of the present invention is without any detectable impurities/contamination of any other previously known crystalline forms of Imatinib mesylate.

In a further aspect, the present application also relates to a pharmaceutical composition comprising crystalline non-needle shaped Form-SA of Imatinib mesylate of the present application and at least one or more pharmaceutically acceptable excipients. Such composition is substantially free of any other previously known crystalline forms of Imatinib mesylate.

Further particular aspects of the invention are detailed in the description part of the specification, wherever appropriate.

### BRIEF DESCRIPTION OF THE DRAWINGS

FIG. 1 is an example of X-ray powder diffraction (“XRPD”) pattern of crystalline Form-SA of Imatinib mesylate

FIG. 2 is an example of a microscopic view of Form-SA of Imatinib mesylate

FIG. 3 is an example of a Differential Scanning calorimetry (“DSC”) curve of Form-SA of Imatinib mesylate

### ABBREVIATIONS

| | |
|---|---|
| API | Active Pharmaceutical Ingredient |
| DIPE | Di-Isopropyl Ether |
| DPE | Di-Propyl Ether |
| DSC | Differential Scanning Calorimetry |
| HPLC | High-Performance Liquid Chromatography |
| IPA | Iso-Propyl Alcohol |
| MTBE | Methyl Tert-Butyl Ether |
| RPM | Rotations Per Minute |
| THF | TetraHydroFuran |
| XRPD | X-Ray Powder Diffraction |

### DETAILED DESCRIPTION

As set forth herein, embodiments of the present invention relate to a reproducible and efficient process for preparation of crystalline non-needle shaped Form-SA of Imatinib mesylate (I) in high yield. Crystalline Form-SA of Imatinib mesylate obtained by the process of the present invention is found to be substantially pure, stable and non-needle shaped.

In one embodiment of the present application, it provides a process for the preparation of crystalline non-needle shaped Form-SA of Imatinib mesylate (I), comprising the steps of:

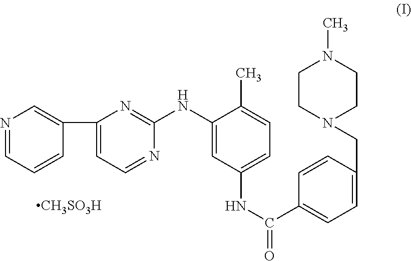

a) Providing a solution of Imatinib base in the mixture of isopropyl alcohol and a cyclic hydrocarbon or ether solvent;
b) Stirring the reaction mass at RPM of not less than 100 rotations per minute;
c) Adding solution of methane sulfonic acid and isopropyl alcohol to the reaction mass in time duration of not less than 30 mins;
d) Heating the reaction mass to a temperature ranging between 60-75° C.;
e) Cooling the reaction mass to ambient temperature of 25-30° C. in time duration of not less than 4 hours.
f) Recovering the non-needle shaped crystalline Form-SA.
   The individual steps of the process according to the present invention for preparing crystalline non-needle shaped Form-SA of Imatinib mesylate (I) are detailed separately herein below.
   Step a) comprises providing a solution of Imatinib base in the mixture of isopropyl alcohol and a cyclic hydrocarbon or ether solvent;

Imatinib base from any source is provided as a solution in the mixture of isopropyl alcohol (IPA) and a cyclic hydrocarbon or ether solvent. The cyclic hydrocarbon solvent to be used in this reaction may be selected from C5-C7 hydrocarbon like cyclopentane, cyclohexane and cycloheptane; and ether solvent may be selected from diethyl ether, DIPE, DPE, MTBE or THF.

The ratio of amount of isopropyl alcohol and the cyclic hydrocarbon/ ether solvent, used for this reaction is very important for the desired end product characteristics. Preferably, isopropyl alcohol and cyclic hydrocarbon or ether solvent are used in the ratio ranging from 7:3 to 8:2 (v/v). In a particular embodiment, for 80 ml IPA, 20 ml of cyclohexane was used to prepare the solvent mixture for providing a solution of Sorafenib base. In another particular embodiment, for 4200 ml IPA, 1800 ml of DIPE was used to prepare the solvent mixture for providing a solution of Sorafenib base.

Step b) comprises stirring the reaction mass at RPM of not less than 100 rotations per minute;

The reaction mass obtained from step a) is subjected to stirring. It has been found by the inventors of this application that stirring plays a very critical role in obtaining the desired characteristics of the end product i.e. Form-SA of Imatinib mesylate. For a commercially viable process to obtain Form SA of Imatinib mesylate, stirring shall be performed at RPM of at least not less than 100 rotations per minute.

In a preferred embodiment stirring of the reaction mass is performed at RPM of about 130-150 rotations per minute for commercial scale batches. For laboratory scale batches, RPM of 200-250 rotations per minute shall be maintained while stirring Importance of the role played by rate of stirring of the reaction, can be gauged from the fact that, when stirring RPM of less than 100 rotations per minute is used in this reaction, the end product Imatinib mesylate is not obtained as Form-SA i.e. the end product is not obtained in pure form and shows the presence of needle shaped crystals and other impurities. Thus controlled stirring conditions of not less than 100 rotations per minute shall be maintained throughout this reaction to obtain the desired end product as crystalline non-needle shaped Form-SA of Imatinib mesylate.

Step c) comprises adding solution of methane sulfonic acid and isopropyl alcohol to the reaction mass in time duration of not less than 30 mins;

To the properly stirred reaction mass of step b), while continuing the stirring a solution of methane sulfonic acid and isopropyl alcohol is added in time duration of not less than 30 mins. In this step, methanesulfonic acid and isopropyl alcohol are used in the ratio ranging from 1:1 to 1:3 (w/v-methanesulfonic acid: isopropyl alcohol). In one of the particular embodiment methanesulfonic acid and isopropyl alcohol are used in the ratio of 1:1, for e.g. 79.10 g of methanesulfonic acid solution with 80 ml of isopropyl alcohol was added to the reaction mass. This step is carried out at room temperature.

The addition of solution of methane sulfonic acid and isopropyl alcohol to the reaction mass obtained in step b) shall be performed slowly in time duration of not less than 30 mins. If a faster addition of this solution is done to the reaction mass obtained in step b) then the end product does not comply to the characteristics of crystalline non-needle shaped Form-SA of Imatinib mesylate.

Step d) comprises heating the reaction mass to a temperature ranging between 60-75° C.;

After the completion of the addition of solution of methane sulfonic acid and isopropyl alcohol to the reaction mass, in step c) the reaction mass is heated to a temperature of 60-75° C., along with the continuous controlled stirring. In one of the preferred embodiment the reaction mass is preferably heated to a temperature of 70-75° C. The reaction mass is maintained at this raised temperature for time duration of 2-5 hours, depending upon the progress of the reaction as is monitored intermittently during the reaction.

Step e) comprises cooling the reaction mass to ambient temperature of 25-30° C. in time duration of not less than 4 hours.

The heated reaction mass obtained from step d) is cooled gradually to an ambient temperature of 25-30° C. in time duration of not less than 4 hours. In one of the particular embodiment the reaction temperature was allowed to cool down from 70° C. to 27° C. in time duration of 5 hours. To maintain the characteristic properties of the end product to be obtained as crystalline non-needle shaped Form-SA of Imatinib mesylate the cooling of the reaction mass shall not be performed forcefully or abruptly at a faster rate. The cooling rate may be required to be controlled mechanically in atmospheres where the normal room temperature is sub-25° C., so as to avoid abrupt cooling.

Step f) comprises recovering the crystalline non-needle shaped form of Imatinib mesylate designated as Form-SA.

The reaction mass obtained from step e) is filtered and given washing with an alcoholic solvent. Alcoholic solvent for this reaction step may be selected from C1-C4 alcohol. In one of the preferred embodiment, the filtered reaction mass obtained in this step is given washing with IPA. The amount of alcoholic solvent used in this reaction ranges from 1-3 times (w/v) w.r.t. the amount of Imatinib base used initially in step a). The material obtained after the alcoholic solvent washing is then dried under reduced pressure conditions to recover the crystalline non-needle shaped form of Imatinib mesylate designated as Form-SA. According to the requirement, raised temperature of 70-140° C. may also be utilized during the drying of the end product under reduced pressure conditions. Reduced pressure conditions may be suitably employed by a person skilled in the art.

Process of recovering the crystalline non-needle shaped Form-SA of Imatinib mesylate may further comprise processes but not limited to conventional processes including scrapping and if required filtering from slurry which may be carried out at room temperature for the suitable durations to retain the crystalline form characteristics of Form-SA.

The process related impurities that appear in the impurity profile of Imatinib mesylate may be substantially removed by the process of the present invention resulting in the formation of crystalline Form-SA of high purity. The merit of the process according to the present invention resides in that—product isolated after drying is directly obtained as crystalline non-needle shaped Form-SA of Imatinib mesylate.

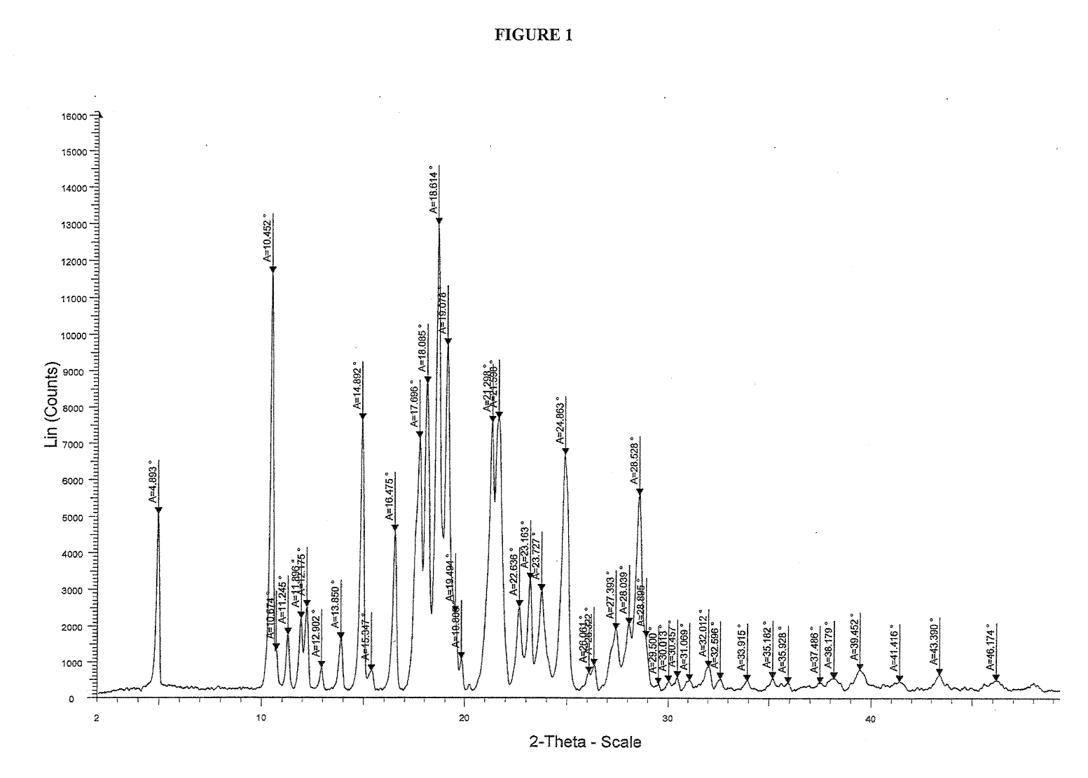

The crystalline non-needle shaped Form-SA of Imatinib mesylate described herein may be characterized by X-ray powder diffraction pattern (XRPD) and Thermal techniques such as differential scanning calorimetry (DSC) analysis. The samples of crystalline non-needle shaped Form-SA of Imatinib mesylate were analyzed by XRPD on a Bruker AXS D8 Advance Diffractometer using X-ray source—Cu Kα radiation using the wavelength 1.5418 Å and lynx Eye detector. DSC was done on a Perkin Elmer Pyris 7.0 instrument. Illustrative examples of analytical data for the crystalline non-needle shaped Form-SA of Imatinib mesylate obtained in the examples are set forth in the FIGS. 1-3.

Another embodiment of the present invention provides crystalline non-needle shaped

Form-SA of Imatinib mesylate obtained by the process according to the present invention. Form-SA of Imatinib mesylate is found adequately stable to handle and store for longer time (at least up to more than 6 months) without any significant or measurable change in its morphology and physicochemical characteristics. Crystalline non-needle shaped Form-SA of Imatinib mesylate obtained according to the process of the present invention is substantially pure i.e. it is found to have final API HPLC purity of more than 99.8% w/w, with moisture content of not more than 0.5%.

Crystalline non-needle shaped Form-SA of Imatinib mesylate, is a non-hygroscopic crystalline solid, which is characterized by—
i. X-ray powder diffraction pattern comprising of at least five 2θ° peaks selected from 10.67, 12.90, 15.34, 19.49, 19.80, 26.06, 26.32 and 28.89±0.05 2θ°;
ii. X-ray powder diffraction pattern with absence of 2θ° peak at about 25.08 2θ°;
iii. Non-needle shaped crystals;
iv. Particle size distribution of d₉₀ ranging from 90 to 130 μm.

The crystalline Form-SA of Imatinib mesylate as obtained by the process of the present invention is without any detectable impurities/contamination of any other previously known crystalline forms of Imatinib mesylate.

The process of the present invention provides non-needle shaped crystals up to extent of 100%, with the absence of any needle-shaped crystals in the obtained end product i.e. crystalline Form-SA.

In one of the embodiments the crystalline Form-SA of Imatinib mesylate shows plate, flake, Lath or equant type of crystal shapes, having absence of any trace of needle type crystalline material. The visual observation of the crystalline material obtained by process of the present invention provides physical shape information about the crystals to be present as plates, stacked plates, agglomerate of plates, spherulite or radial clusters, conglomerate of different shapes but no presence of any acicular material which includes any needle type particle or pointed slender material is observed.

In a further embodiment according to this specification, the invention also relates to a composition containing Crystalline non-needle shaped Form-SA of Imatinib mesylate, which is substantially free of any other known forms of Imatinib mesylate.

The Crystalline non-needle shaped Form-SA of Imatinib mesylate obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin.

In one embodiment of the present invention, it also includes premix comprising one or more pharmaceutically acceptable excipients in the range of 1 to 50% w/w with crystalline non-needle shaped Form-SA of Imatinib mesylate, while retaining the crystalline nature of the premix.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising crystalline non-needle shaped Form-SA of Imatinib mesylate according to the present application include, but are not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of crystalline non-needle shaped Form-SA of Imatinib mesylate of the present application may also comprise to include the pharmaceutically acceptable carriers used for the preparation of solid dispersion, wherever utilized in the desired dosage form preparation.

### EXAMPLES

### Example-01

### Process for Preparation of Crystalline Non-Needle Shaped Form-SA of Imatinib Mesylate Using IPA and C-6 Cyclic Hydrocarbon Solvent

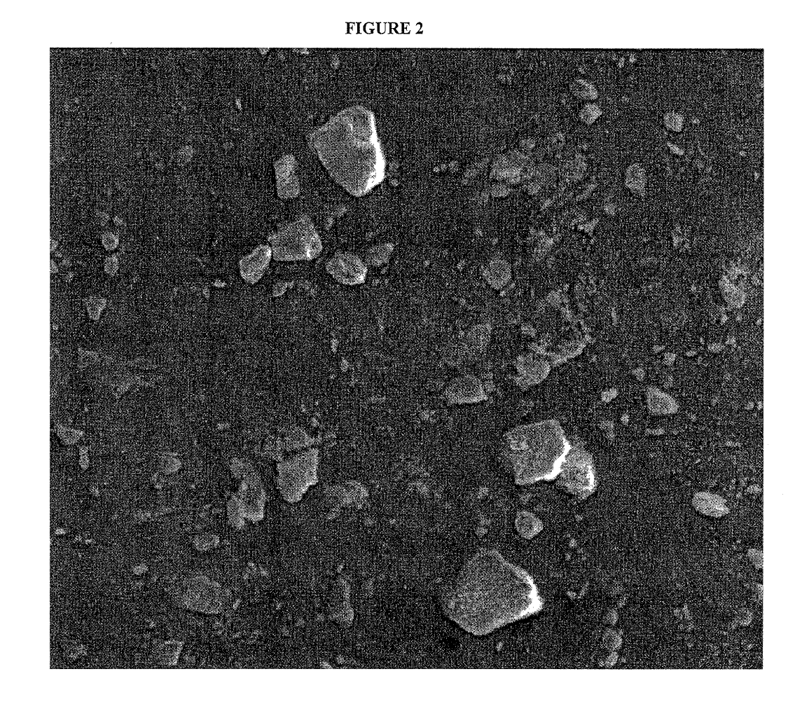

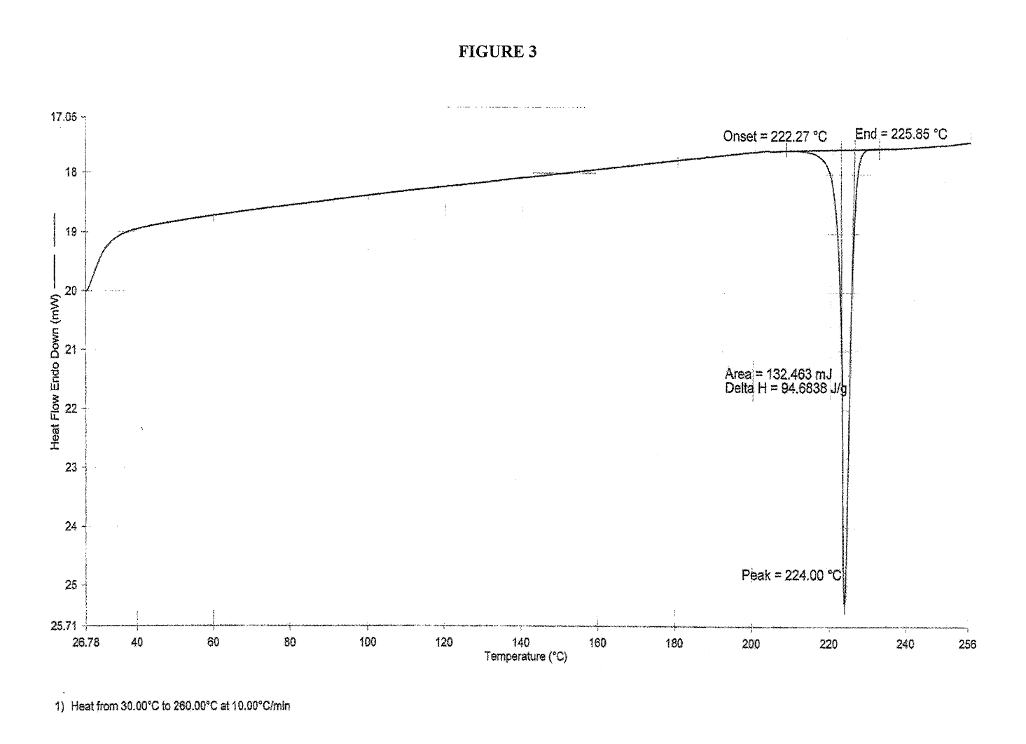

5.0 g of Imatinib base was added to a mixture of 80 ml of isopropyl alcohol & 20 ml of cyclohexane. Stirring of the reaction mixture was performed at RPM of 200 rotations per minute. To the stirring reaction mass was added the solution of methanesulfonic acid (0.985 g) and 2 ml of isopropyl alcohol within 30 mins at RT. After the completion of this addition, reaction mass was heated to 75° C. and this raised temperature along with controlled stirring at 200 RPM was maintained for 3 hrs. Then the reaction mass was slowly cooled to 25° C. in 4 hrs. The reaction mass was then filtered, washed with 10 ml of isopropyl alcohol (IPA) and dried under vacuum at 120-130° C. for 24 hrs, to obtain the crystalline non-needle shaped Form-SA of Imatinib mesylate having XRPD similar to FIG. 1, Microscopic view similar to FIG. 2 and DSC thermogram similar to FIG. 3.

Yield: 5.2 g; HPLC purity: 99.85%; Moisture Content 0.40% (by KF)

### Example-02

### Process for Preparation of Crystalline Non-Needle Shaped Form-SA of Imatinib Mesylate Using IPA and Ether Solvent

300.0 g of Imatinib base was added to a mixture of 4200 ml of isopropyl alcohol & 1800 ml of DIPE. Stirring of the reaction mixture was performed at RPM of 140 rotations per minute. To the stirring reaction mass was added the solution of methanesulfonic acid (59.10 g) and 150 ml of isopropyl alcohol within 30 mins at RT. After the completion of this addition, reaction mass was heated to 70° C. and the raised temperature along with controlled stirring at 140 RPM was maintained for 3 hrs. The reaction mass was slowly cooled to 27° C. in 5 hrs. The reaction mass was then filtered, washed with 600 ml of isopropyl alcohol (IPA) and dried under vacuum at 80° C. for 24 hrs, to obtain the crystalline non-needle shaped Form-SA of Imatinib mesylate having XRPD similar to FIG. 1 and DSC thermogram similar to FIG. 3.

Yield: 329.0 g; HPLC purity: 99.89%; Moisture Content 0.42% (by KF)

### Example-03

### Process for Preparation of Crystalline Non-Needle Shaped Form-SA of Imatinib Mesylate Using IPA and DPE

400.0 g of Imatinib base was added to a mixture of 5600 ml of isopropyl alcohol & 2400 ml of DPE. Stirring of the reaction mixture was performed at RPM of 130 rotations per minute. To the stirring reaction mass was added the solution of methanesulfonic acid (79.10 g) and 80 ml of isopropyl alcohol within 30 mins at RT. After the completion of this addition, reaction mass was heated to 70° C. and the raised temperature along with controlled stirring at 130 RPM was maintained for 3 hrs. Then the reaction mass was slowly cooled to 25° C. in 5 hrs. The reaction mass was then filtered, washed with 800 ml of isopropyl alcohol (IPA) and dried under vacuum at 80° C. for 24 hrs, to obtain the crystalline non-needle shaped Form-SA of Imatinib mesylate having XRPD similar to FIG. 1 and DSC thermogram similar to FIG. 3.

Yield: 418.0 g; HPLC purity: 99.92%; Moisture Content 0.36% (by KF)

While the foregoing pages provide a detailed description of the preferred embodiments of the invention, it is to be understood that the description and examples are illustrative only of the principles of the invention and not limiting. Furthermore, as many changes can be made to the invention without departing from the scope of the invention, it is intended that all material contained herein be interpreted as illustrative of the invention and not in a limiting sense.

1) Process for the preparation of crystalline non-needle shaped Form-SA of Imatinib mesylate (I), comprising the steps of: 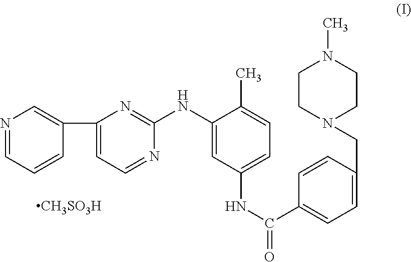
a) Providing a solution of Imatinib base in the mixture of isopropyl alcohol and a cyclic hydrocarbon or ether solvent;
b) Stirring the reaction mass at RPM of not less than 100 rotations per minute;
c) Adding solution of methane sulfonic acid and isopropyl alcohol to the reaction mass in time duration of not less than 30 mins;
d) Heating the reaction mass to a temperature ranging between 60-75° C.;
e) Cooling the reaction mass to ambient temperature of 25-30° C. in time duration of not less than 4 hours. f) Recovering the non-needle shaped crystalline Form-SA.

2) Process for the preparation of crystalline non-needle shaped Form-SA of Imatinib mesylate, according to claim 1, wherein cyclic hydrocarbon solvent is selected from C5-C7 hydrocarbon and ether solvent is selected from diethyl ether, DIPE, DPE, MTBE or THF.

3) Process for the preparation of crystalline non-needle shaped Form-SA of Imatinib mesylate, according to claim 1, wherein in step a) isopropyl alcohol and cyclic hydrocarbon or ether solvent are used in the ratio ranging from 7:3 to 8:2 (v/v).

4) Process for the preparation of crystalline non-needle shaped Form-SA of Imatinib mesylate, according to claim 1, where in step c) methanesulfonic acid and isopropyl alcohol are used in the ratio ranging from 1:1 to 1:3 (w/v-methanesulfonic acid: isopropyl alcohol).

5) Process for the preparation of crystalline non-needle shaped Form-SA of Imatinib mesylate, according to claim 1, wherein stirring of the reaction mass is performed at RPM of 130-150 rotations per minute for commercial scale batches and at RPM of 200-250 rotations per minute for laboratory scale batches.

6) Process for the preparation of crystalline non-needle shaped Form-SA of Imatinib mesylate, according to claim 1, wherein step f) further comprises the steps of:
i. Filtering the reaction mass;
ii. Washing with an alcoholic solvent;
iii. Drying under reduced pressure conditions to recover the crystalline non-needle shaped form of Imatinib mesylate designated as Form-SA.

7) Crystalline non-needle shaped Form-SA of Imatinib mesylate obtained by the process according to any of the claims 1 to 6.

8) Crystalline Form-SA of Imatinib mesylate characterized by
i. X-ray powder diffraction pattern comprising of at least five 2θ° peaks selected from 10.67, 12.90, 15.34, 19.49, 19.80, 26.06, 26.32 and 28.89±0.05 2θ°;
ii. X-ray powder diffraction pattern with absence of 2θ° peak at about 25.08 2θ°;
iii. Non-needle shaped crystals;
iv. Particle size distribution of d₉₀ ranging from 90 to 130 μm.

9) Substantially pure crystalline non-needle shaped Form-SA of Imatinib mesylate with HPLC purity of at least 99.8% and moisture content less than 0.5% w/w.

10) A pharmaceutical composition comprising non-needle shaped Form-SA of Imatinib mesylate obtained by the process according to any of the preceding claims, and at least one or more pharmaceutically acceptable excipients.
